(19) United States
(12) Patent Application Publication
(10) Pub. No.: US 2012/0143040 A1
(43) Pub. Date: Jun. 7, 2012

(54) PATIENT COMMUNICATION AND MONITORING IN MAGNETIC RESONANCE IMAGING SYSTEMS

(75) Inventors: Chinmoy Goswami, Waukesha, WI (US); Baldev Ahluwalia, Waukesha, WI (US); Daniel Lynch, Waukesha, WI (US)

(73) Assignee: GENERAL ELECTRIC COMPANY, Schenectady, NY (US)

(21) Appl. No.: 12/961,118

(22) Filed: Dec. 6, 2010

(51) Int. Cl. A61B 5/055

(52) U.S. Cl. 600/410

(57) ABSTRACT

The present embodiments provide an improved system and method for patient communication during MR procedures. In one embodiment, a magnetic resonance imaging (MRI) system is provided. The system includes a scanner having an opening configured to receive a patient and being operable to perform a magnetic resonance imaging sequence. The system also includes a visual communication device disposed in the opening of the scanner and being operable to capture an image of the patient and transmit the image to circuitry outside of the scanner.

### BACKGROUND OF THE INVENTION

The subject matter disclosed herein relates to communication and data acquisition in magnetic resonance imaging systems, and more specifically to communication with a patient while the patient is undergoing an examination.

Magnetic resonance (MR) imaging techniques are used for a wide range of diagnostic purposes in medicine. In general, such techniques rely on interaction between gyromagnetic material in tissues, that are affected by magnetic fields, and controlled fields that combine to encode locations of the tissues, and radio frequency pulses that perturb the materials, resulting in magnetic resonance echoes when the materials return to their equilibrium magnetization. Data from the radio frequency echo signals is acquired and is used to produce an MR image. During the acquisition of the RF signal, patient movement can result in image artifacts. One approach to minimize such movement is for the patient to hold their breath.

Breath-hold imaging techniques are used in about 25% of all MR imaging exams. During a breath-hold exam, patients are asked to repeatedly hold their breath for between 10 and 25 seconds, during a total exam time of about 30 minutes. As noted above, the diagnostic quality of the resulting MR images obtained during such exams relies heavily on the patient's ability to remain still, i.e., hold their breath. Accordingly, the synchrony of performing an MR scan and instructing the patient when to hold their breath can be of great importance to the success of the imaging procedure. Typically, a patient is told to hold their breath through some form of verbal communication from the technologist performing the imaging exam. As an example, there may be speakers or an intercom disposed proximate the MR scanner that are used to convey the auditory instructions from the technician to the patient. Unfortunately, the MR scanner can generate noise that may interfere with the patient's ability to hear the technician's instructions, and the electrical wiring required for auditory communication can be undesirable in the presence of the magnetic field generated in the immediate vicinity of the scanner. Moreover, it can be difficult for the patient to judge the duration of the breath hold when no indicia have been provided (e.g., to release their held breath).

Accordingly, there is a need for an improved method of communication between the technologist performing the imaging routine and the patient undergoing MR imaging examinations.

### BRIEF DESCRIPTION OF THE INVENTION

As noted above, the present embodiments provide an improved system and method for patient communication during MR procedures. In one embodiment, a magnetic resonance imaging (MRI) system is provided. The system includes a scanner having an opening configured to receive a patient and operable to perform a magnetic resonance imaging sequence and a visual communication device disposed in the opening of the scanner and being operable to transmit visual information originating from circuitry outside of the scanner to the opening.

In another embodiment, an MRI system is provided. The system includes a scanner having an opening configured to receive a patient and being operable to perform a magnetic resonance imaging sequence. The system also includes a visual communication device disposed in the opening of the scanner and being operable to transmit visual information originating from circuitry outside of the scanner to the patient while the patient is in the scanner.

In a further embodiment, an MRI communication system is provided. The system includes a visual communication device configured to be placed in a patient opening of an MRI scanner. The visual communication device is operable to transmit visual information originating from circuitry outside of the MRI scanner to a patient in the patient opening.

In another embodiment, an image captured using an MRI communication system is provided. The communication system includes a visual communication device configured to be placed in a patient opening of an MRI scanner, the visual communication device being operable to capture the image in the patient opening.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical illustration of an MRI system that is configured to allow an imaging technician to communicate with and/or monitor a patient while the patient is in the MR scanner;

FIG. 2 is a diagrammatical illustration of an embodiment of the visual communication device of FIG. 1, wherein the device includes a panel disposed within a patient opening of the MR scanner and hinged so as to allow the panel to extend and retract from the endbell of the scanner;

FIG. 3 is a diagrammatical illustration of an embodiment of the visual communication device disposed within the MR scanner of FIG. 1, the device having a panel with patient instructions;

FIG. 4 is a diagrammatical illustration of an embodiment of the visual communication device disposed within the MR scanner of FIG. 1, the device being configured to project an image within the MR scanner so as to allow a patient to view the image while in the scanner;

FIG. 5 is a diagrammatical illustration of an embodiment of the visual communication device disposed within the MR scanner of FIG. 1, the device being configured to capture an image of the patient while the patient is in the MR scanner;

FIG. 6 is a diagrammatical illustration of an embodiment of the visual communication device disposed within the MR scanner of FIG. 1, the device being configured to both provide visual instructions to the patient and capture an image of the patient while the patient is in the MR scanner;

FIG. 7 is a diagrammatical illustration of an embodiment of the visual communication device disposed within the MR scanner of FIG. 1, the device being attached to a low profile carriage assembly (LPCA) and configured to both provide visual instructions to the patient and capture an image of the patient while the patient is in the MR scanner; and

FIG. 8 is an illustrative example of an embodiment of an arrangement of patient data, a patient image captured using the visual communication device, and MR images produced using the system of FIG. 1.

### DETAILED DESCRIPTION OF THE INVENTION

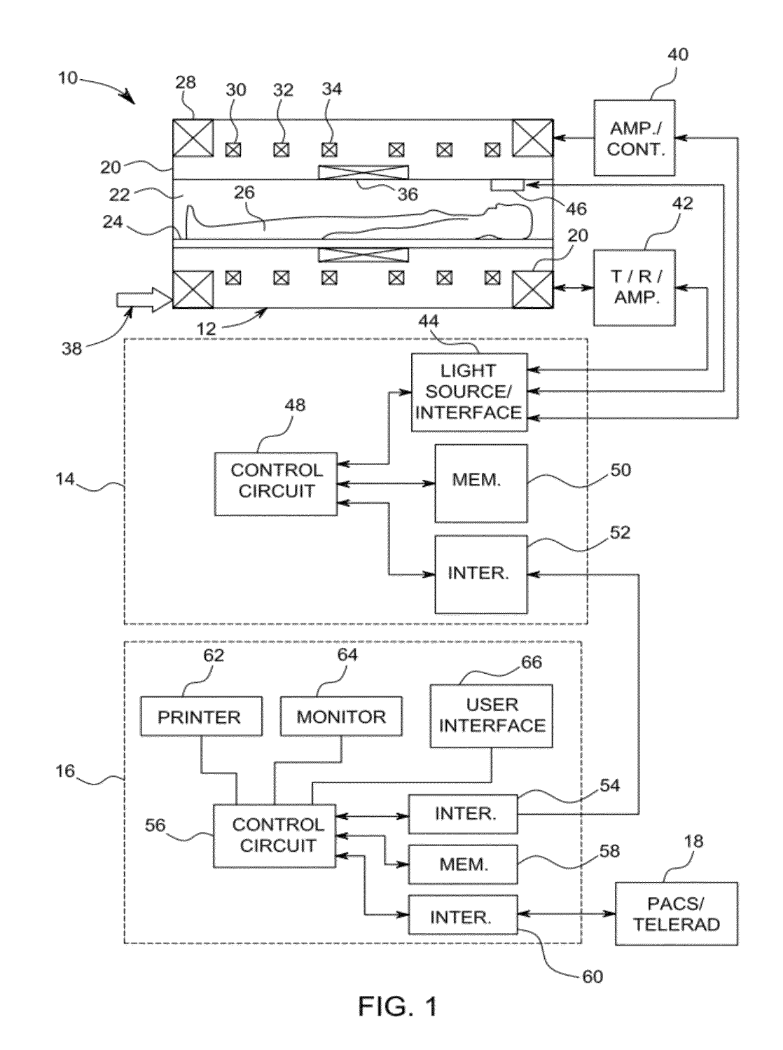

Referring to FIG. 1, an MRI system 10 is illustrated schematically as including a scanner 12, scanner control circuitry 14, and system control circuitry 16. According to the embodiments described herein, the MRI system 10 is generally configured to perform MR imaging, such as in sequences where a patient performs a breath-hold, and the system collects MR data from gyromagnetic nuclei within the patient. System 10 may additionally include remote access and storage systems or devices as picture archiving and communication systems (PACS) 18, or other devices such as teleradiology equipment so that data acquired by the system 10 may be stored, and where desired, accessed on- or off-site. Moreover, the PACS/teleradiology 18 may enable an off-site user, such as a remote radiologist, to visibly communicate with a patient while the patient is in the scanner 12 via one or more visual communication devices described below. Further, while visual communication devices in accordance with present embodiments may be implemented in any suitable scanner or detector, such as an open MRI system, in the illustrated embodiment, the system 10 includes a full body scanner 12 having a housing 20 through which an opening or bore 22 is formed. A table 24 is moveable into the bore 22 to permit a patient 26 to be positioned for imaging selected anatomies within the patient. In some embodiments, as described below, one or more visual communication devices may move in concert with or may be directly attached to the table 24 to allow the imaging personnel to communicate with the patient 26 during all phases of operation of the system 10.

Scanner 12 includes a series of associated coils for producing controlled magnetic field and for detecting emissions (radio frequency echoes) from gyromagnetic material within the subject being imaged. A primary magnet coil 28 is provided for generating a primary magnetic field that is generally aligned with the bore 22. A series of gradient coils 30, 32, and 34 are configured to permit controlled magnetic gradient fields to be generated during examination sequences. The controlled magnetic gradient fields generally encode positional information into at least a portion of gyromagnetic nuclei (i.e., ¹H) within the patient 26. A radio frequency (RF) coil 36 is provided for generating RF pulses for exciting the gyromagnetic material, such as for spin preparation, relaxation weighting, spin perturbation or slice selection. A separate receiving coil or the same RF coil 36 may receive MR signals from the gyromagnetic material during examination sequences. For example, a receiving RF coil may collect RF signals from the gyromagnetic nuclei while the patient 26 performs a breath-hold.

During such an examination, the various coils of scanner 12 are controlled by external circuitry to generate the desired field and pulses, and to read emissions from the gyromagnetic material in a controlled manner. The external circuitry, such as scanner control circuitry 14 and system control circuitry 16, may be disposed in another room, or at an even greater distance from the scanner 12. In the illustrated embodiment, a main power supply 38 is provided for powering the primary field coil 28. A driver circuit 40 is provided for pulsing the gradient field coils 30, 32, and 34. Such circuitry typically includes amplification and control circuitry for supplying current to the coils as defined by digitized pulse sequences output by the scanner control circuit 14. Another control circuit 42 is provided for regulating operation of the RF coil 36. Circuit 42 will typically include a switching device for alternating between the active and passive modes of operation, wherein the RF coil transmits and receives signals, respectively. Circuit 42 also includes amplification circuitry for generating the RF pulses and for processing received magnetic resonance signals.

Scanner control circuit 14 includes an interface circuit 44 which outputs signals for driving the gradient field coils 30, 32, 34 and the RF coil 36 and for receiving the data representative of the MR signals produced in examination sequences. In some embodiments, the interface circuit 44 may synchronize such output signals to the gradient field coils 30, 32, 34 (or other scanner coils) with one or more visual indications (e.g., breath-hold instructions) to the patient 26 provided by a visual communication device 46. Accordingly, the interface circuit 46 is in communication with the visual communication device 46 and may include one or more light sources and related optical communication features (e.g., fiber optic lines). Moreover, the visual communication device 46 may allow the user to ascertain positional information about the patient 26, such as the positioning of anatomies of interest of the patient 26 relative to the coils of the scanner 12.

The interface circuit 44 is coupled to a control circuit 48. The control circuit 48 executes the commands for driving circuit 40, circuit 42, and the visual communication device 46 based on defined protocols selected via system control circuit 16. Control circuit 48 also serves to receive MR signals produced from imaging sequences, as well as images of the patient 26 that may be captured by the visual communication device 46. The control circuit 48 performs subsequent processing on the collected data and/or images before transmitting the data to system control circuit 16. As an example, the control circuit 48 may associate the collected MR data (which may be processed data such as one or more MR images) with one or more photographic images of the patient 26 captured by the visual communication device 46. The captured and/or processed images may be stored on one or more memory circuits 50 of the scanner control circuitry 14. The memory circuits 50 also store configuration parameters (e.g., breath-hold instruction and data collection synchronization parameters), pulse sequence descriptions, examination results, and so forth, during operation. Interface circuit 52 is coupled to the control circuit 48 for exchanging data between scanner control circuit 14 and system control circuit 16. Such data will typically include selection of specific examination sequences to be performed, configuration parameters of these sequences, acquired data, and one or more photographic images of the patient 26. The acquired data may be transmitted in raw or processed form from scanner control circuit 14 for subsequent processing, storage, transmission and display.

System control circuit 16 includes an interface circuit 54 which receives data (e.g., configuration, MR data, patient images) from the scanner control circuit 14 and transmits data and commands back to the scanner control circuit 14. The interface circuit 54 is coupled to a control circuit 56 which may include one or more processors in a multi-purpose or application specific computer or workstation. Control circuit 56 is coupled to a memory circuit 58 to store programming code for operation of the MRI system 10 and to store the processed image data for later reconstruction, display and transmission, for example along with real-time and/or captured images of the patient 26, which will be discussed in detail below. An additional interface circuit 60 may be provided for exchanging image data, configuration parameters, and so forth with external system components such as the PACS/teleradiology system 18. Finally, the system control circuit 56 may include various peripheral devices for facilitating operator interface and for producing hard copies of the captured and/or reconstructed images. In the illustrated embodiment, these peripherals include a printer 62, a monitor 64, and user interface 66 including devices such as a keyboard or a mouse.

As noted above, scanner 12 and control circuit 48 provide control signals to produce magnetic fields and RF pulses in a controlled manner to excite and encode specific gyromagnetic material within the patient 26. The scanner 12 and control circuit 48 also sense the signals emanating from such material and create an image of the material being scanned. For example, in certain embodiments, the scan may be performed while the patient 26 performs a breath-hold. The breath-hold may be initiated by the user performing the scans, for example by providing a visual indication to the patient 26 via the visual communication device 46, or may be initiated automatically by control circuitry (e.g., control circuit 48 and/or control circuit 56) through the visual communication device 46 in synchronization with pulse sequences performed by the coils of the scanner 12.

It should be noted that the MRI system described herein is merely intended to be exemplary, and other system types, such as so-called “open” MRI systems may also be used. Similarly, such systems may be rated by the strength of their primary magnet, and any suitably rated system capable of carrying out the data acquisition and processing described below may be employed. Keeping this in mind, varying embodiments of the visual communication device in accordance with present embodiments are described herein. In a general sense, the visual communication device may be configured to transmit one or more visual instructions to the patient 26, and may also be configured to capture substantially real-time images of the patient 26 as the patient 26 enters and/or is inside the scanner 12. Thus, the visual communication device is configured to be substantially immune to interference (e.g., RF interference) from the MR scanner 12 and its associated features, and will not substantially affect imaging or image quality.

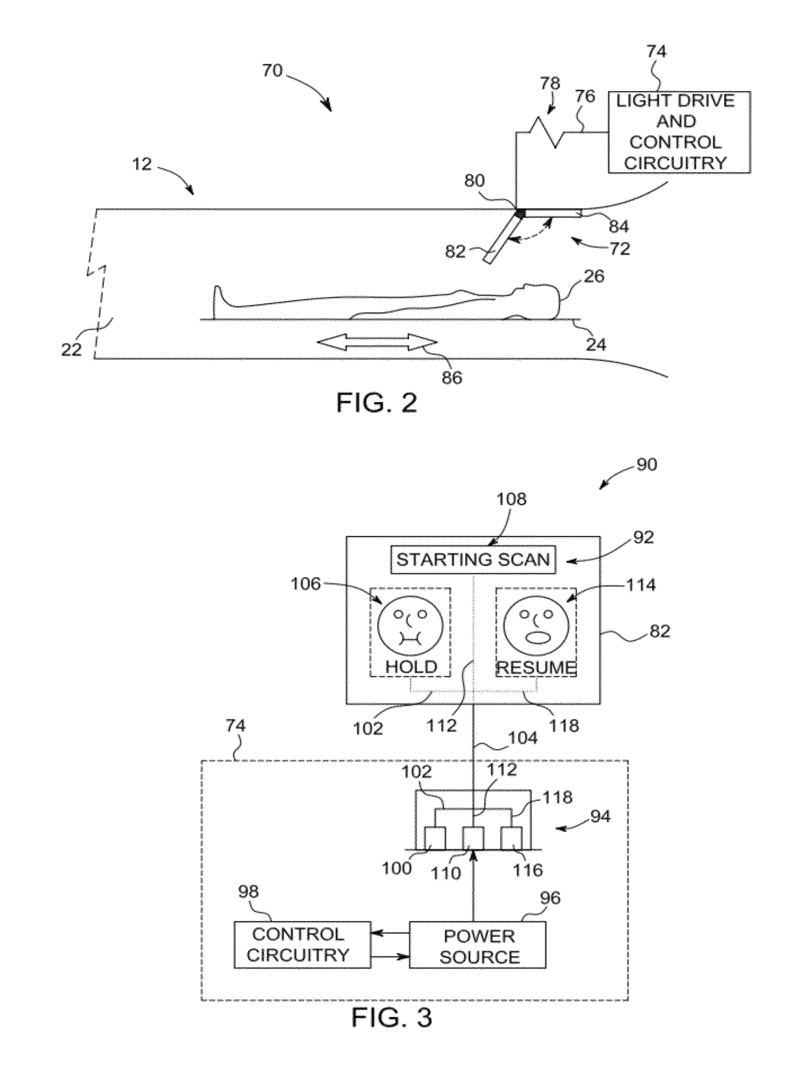

One embodiment by which instructions may be communicated to the patient 26 is illustrated in FIG. 2, which depicts an embodiment of a system 70 having a visual communication device 72 that is moveable within the MR scanner 12. The visual communication device 72 is connected to light drive and control circuitry 74, which includes a light drive and one or more light sources that are controlled by control circuitry. As an example, the control circuitry may be the scanner control circuitry 14 and/or the system control circuitry 16 of FIG. 1. One or more fiber optic lines 76 may transmit light generated at the light drive and control circuitry 74 to the visual communication device 72. It should be noted that the light provided to the visual communication device 72 may be produced at an area disposed at a distance from scanner 12 so as to avoid interference from the scanner 12, which is depicted generally by interface 78. Thus, the light drive and control circuitry 74 may be disposed at a distance, in another room, and/or at another facility away from scanner 12.

In the illustrated embodiment, the visual communication device 72 includes a joint 80 which hinges a panel 82 between a first position and a second position, which is illustrated as panel 84. The first position (panel 82) may be a position at which the patient 26 is able to receive visual instructions, such as breath-hold instructions. As discussed generally above, due to the positioning of the panel 82 directly within the scanner 12, the panel 82 may be constructed so as to include only materials that are substantially immune to interference from the MR scanner 12 (i.e., RF interference). Examples of such materials may include natural and synthetic plastics such as acrylics, glass such as silica-based glass, and so forth. An embodiment of such a panel is discussed with respect to FIG. 3 below. During operation of the scanner 12, the patient 26 may be displaced within the patient opening (bore 22) by moving the table 24. The movement of the table 24, depicted generally by dual-headed arrow 86, may be matched by rotational movement of the panel 82 from its first position to its second position. In this way, the panel 82 moves in the direction of patient displacement, with panel 82 moving from the first position to the second position (panel 84) as the patient 26 exits the bore 22, and moving from its second position (panel 84) to its first position (panel 82) as the patient 26 enters the bore 22.

In the embodiment illustrated in FIG. 2, the panel 82 is designed so as to allow the patient 26 to receive visual instructions while situated inside the scanner 12. An example of such a display system 90 having the panel 82 for providing patient instructions is illustrated in FIG. 3. It should be noted that while the present approaches are discussed in the context of breath-hold examinations, that such a context is merely to facilitate explanation of the present approaches, and that the approaches described herein are applicable to a variety of communications provided to the patient area of the scanner 12.

Specifically, the panel 82 is illustrated as including a plurality of segments 92 that are configured to illuminate when light is received. As noted above, in some embodiments, the segments 92 can be constructed directly from the main material of the panel 82, such as by etching. In other embodiments, the segments 92 may be placed on the panel 82 or otherwise inserted into the panel 82. The segments 92 are illuminated upon receiving light from a light source, which in the illustrated embodiment includes a multi-color light emitting diode (LED) 94 that is configured to transmit a separate visible color to each segment 92. However, it should be noted that in other embodiments the light source may be a single-color light source that selectively illuminates each of the segments 92 separately. The multi-color LED 94 is driven by a power source 96, which transmits signals to the multi-color LED 94 to activate selected colors for illumination of one or more of the segments 92. During operation, control circuitry 98, which may be a part of scanner control circuitry 14 and/or system control circuitry 16, may send activation signals to the power source 96 so as to cause the multi-color LED 94 to illuminate one or more of the colors. The selected activation, as noted above, may be performed in synchrony with one or more pulse sequences that are played out on the MR scanner 12. Moreover, in some embodiments, the technician may choose which of the segments 92 are activated. In such embodiments, the control circuitry 98 may or may not be present.

Specifically, in embodiments where the instructions are automatically provided to the patient 26, during a phase of operation of the MR scanner 12 in which data will be acquired, the control circuitry 98 may cause a first LED color 100 contained within the multi-color LED 94 to be activated. Activation of the first LED color 100 causes a first color to be transmitted along a first fiber optic channel 102. The first fiber optic channel 102 then becomes part of a fiber optic bundle 104, which generally includes lines for carrying other colors and may be considered all or a part of fiber optic lines 76 of FIG. 2. The first fiber optic channel 102 then branches off of the fiber optic bundle 104 upon reaching the display 82, and transmits the first color to a breath-hold indication 106. The breath-hold indication 106, as will be appreciated, will generally remain illuminated as MR data is acquired from the patient 26.

So that the patient 26 is aware that MR data is to be collected, the control circuitry 98 may also provide signals so as to cause a scan indication 108 to become illuminated. Specifically, the power source 96 may provide power to the multi-color LED 94 to activate a second LED color 110. The second color that results from the activation may be transmitted along a second fiber optic channel 112, which becomes a part of the fiber optic bundle 104. Upon reaching the display 82, the second fiber optic channel 112 branches off from the bundle 104 and transmits the second color to illuminate the scan indication 108. The scan indication 108 may be a starting scan indication as illustrated, or may simply be an indication throughout the duration of data collection.

When MR data collection is complete, the control circuitry 98 may send signals so as to de-activate the breath-hold indication 106 and the starting scan indication 108. Additionally, the control circuitry 98 may initiate the activation of a resume breathing indication 114. As with the breath-hold indication 106 and the scan indication 108, to activate the resume breathing indication 114 the control circuitry 98 sends signals to the power source 96, which activates a third LED color 116. A third color is then emitted and transmitted along a third fiber optic channel 118. The third fiber optic channel 118 forms a part of the fiber optic bundle 104, and then branches off upon reaching the display 82. The third color is then channeled into the resume breathing indication 114, which is then illuminated.

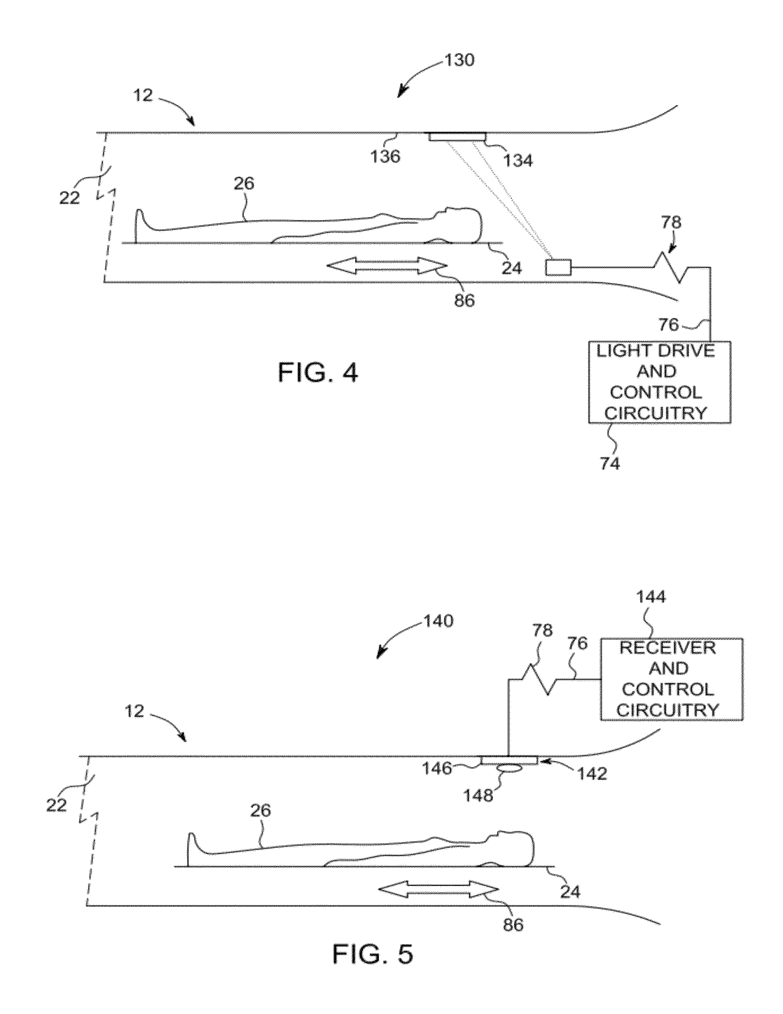

While the system and method described above may generally be implemented by a display, such as display 82, it should be noted that indications may be provided to the patient 26 via projected images. An embodiment of such an approach is illustrated in FIG. 4. Thus, keeping in mind the general operation and provision of indications to the patient 26 discussed with respect to FIG. 3, a system 130 is provided that includes the light drive and control circuitry 74, which is configured to transmit one or more wavelengths of light through fiber optic lines 76. Rather than providing the light directly to an illuminable panel as in FIG. 3, the light is provided to a projection device 132, which is generally configured to receive one or more wavelengths of light and to project an indication using the light to an area that is visible to the patient. As illustrated, the projection device 132 is disposed within the patient opening or bore 22 of the scanner 12. As such, the projection device 132 may consist of materials that are substantially immune to interference from the MR scanner 12. As an example, the projection device 132 may include glass and/or plastic optical components such as fiber optics, etched panels, etched lenses, and so forth.

The projection device 132 may project the patient indications, such as instructions, onto a receiving panel 134 disposed at an area generally visible to the patient 26. The receiving panel 134 may be a mirror, an illuminable panel, or the like. In other embodiments, the indications may simply be projected directly onto an inner surface 136 of the bore 22. In this way, the indications are projected from an area generally behind the patient 26 and onto an area at an opposite diametrical extent from the projection device 132.

As noted above, in addition to or in lieu of providing indications to the patient 26, the present approaches also provide embodiments directed towards capturing one or more images of the patient 26. Of course, the images may be stored on one or more memory circuits of the MR system 10 of FIG. 1, or may be sent to and/or stored on the PACS/teleradiology system 18. The images may be used for determining proper patient alignment for scanning, for determining the level of patient comfort, and for ensuring patient compliance with instructions provided. It should be noted that in embodiments where image acquisition and patient image capture is substantially automatic, that one or more circuits of the MRI system 10 may include image analysis software to determine patient movement (e.g., breath movement) and/or alignment (e.g., body alignment with the scanner coils).

An embodiment of an image capturing system 140 configured to perform such image capturing is illustrated in FIG. 5. Specifically, the visual communication device of system 140 includes an image capturing device 142. The image capturing device 142 is generally configured to collect one or more images of the patient 26 or, in some embodiments, to allow the technician to view the patient 26 in a substantially continuous manner. The image capturing device 142 is connected to receiver and control circuitry 144, which includes an image receiver, such as a camera, and control circuitry that operates to control the acquisition of patient images. Additionally, in some embodiments, the control circuitry may synchronize the image capture process with various phases of operation of the MR scanner 12.

To allow the image capturing device 142 to collect images of the patient 26, the device 142 may include a panel 146 containing a lens 148 that focuses light collected from inside the bore 22 into an image of the patient 26. In this way, the fiber optic line 76, combined with the image capture device 142, may be a fiber optic scope or similar device suitable for viewing the patient 26. Therefore, the image capture device 142 may or may not include the panel 146. In accordance with the present approaches and as illustrated, the image capture device 142 is configured to allow its placement directly inside the bore 22 of the MR scanner 12. Thus, the image capture device 142 is constructed from one or more materials that are substantially immune to interference from the MR scanner 12, such as RF interference.

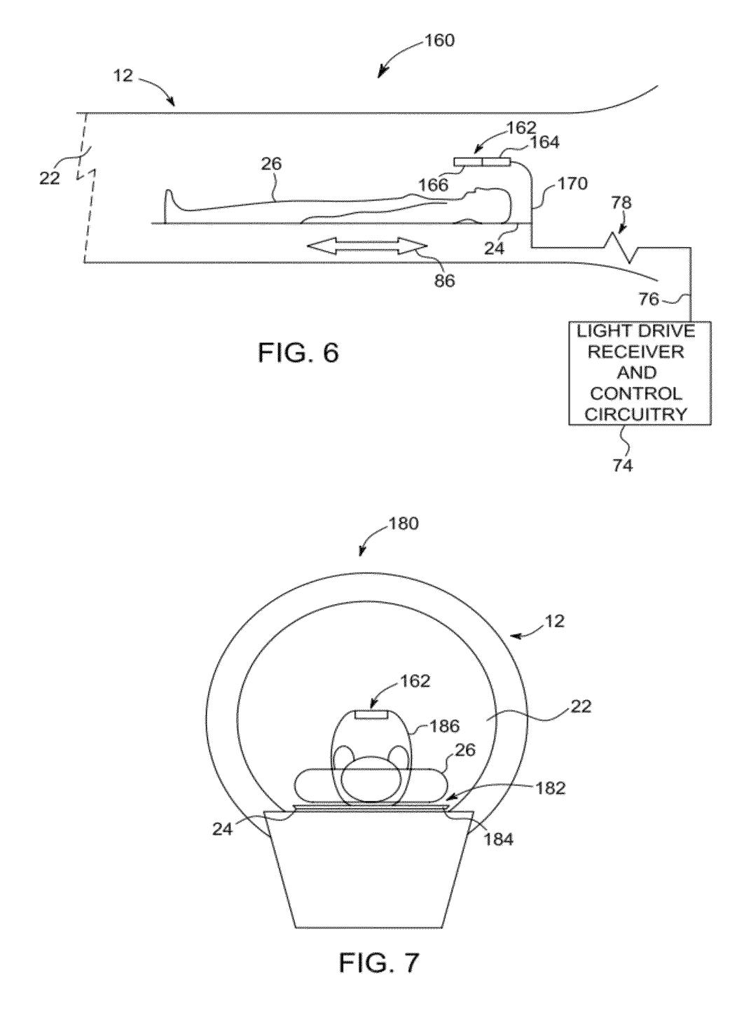

As noted above, it may be desirable to perform such image capturing techniques in addition to providing instructions to the patient 26 while the patient 26 is disposed inside the bore 22. Accordingly, keeping in mind the operation of the systems illustrated in FIGS. 2-5, FIG. 6 illustrates an embodiment of a system 160 including a visual communication device 162 having features for image capture and for providing visual indications to the patient 26. Specifically, the visual communication device 162 includes a combination of an image capture device 164, such as the device 142 described above with respect to FIG. 5, and a display panel 166 for providing patient instructions, such as the panel 82 described with respect to FIGS. 2 and 3.

As illustrated, the image capture device 164, in a similar manner to the display panel 166, is formed as a panel having features for capturing patient images. However, it should be noted that any suitable configuration of the visual communication device 162 is contemplated herein. For example, the visual communication device 162 may include the display panel 166 having a lens that acts as the image capture device 164. In such a configuration, the lens may be disposed proximate the display panel 166 such that light emitted from the display panel 166 may advantageously illuminate the patient 26. Illumination of the patient 26 in this way may allow the image capture device 164 to collect a sufficient amount of light so as to form a patient image suitable for analysis by the technician and/or control circuitry.

In addition to the image capture device 164 and the display panel 166, the system 160 also includes light drive, receiver, and control circuitry 168. The light drive, receiver, and control circuitry 168 includes at least one light source so as to illuminate the display panel 166, the timing of which may be at least partially controlled by control circuitry as described above with respect to FIG. 3. Light drive, receiver, and control circuitry 168 also includes a receiver, such as a camera or other image capturing feature. Of course, the receiver is also connected to control circuitry so as to allow synchrony between patient image capture, MR data acquisition and/or the provision of instructions to the patient via display panel 166. Depending on the particular configuration the system 160, all or a portion of the light drive, receiver, and control circuitry 168 may be disposed in a separate room, or at a separate facility (e.g., in teleradiology applications).

While the visual communication device 162 may be placed at any area within the MR scanner 12 so as to facilitate patient image capture and the provision of instructions or indications to the patient 26, the illustrated embodiment depicts the visual communication device 162 as being attached to the table 24 using a support arm 170. The support arm 170 may be constructed from any number of materials that are suitable for use within the MR scanner 12 (i.e., non-metallic materials and/or materials that are non-magnetic). Further, the support arm 170 may advantageously provide protection (e.g., encasement) for any fiber optic lines (i.e., fiber optic lines 76) that connect the visual communication device 162 to the light drive, receiver, and control circuitry 168. Further, due to the attachment of the support arm 170 to the table 24, the visual communication device 162 may provide indications to the patient 26 and may capture images of the patient 26 while the table 24 is in motion, or regardless of the exact positioning of the table 24 within the MR scanner 12.

While the support arm 170 may provide a number of advantages for the placement of the visual communication device 162 on the table 24 rather than directly onto a surface of the scanner 12, it may be desirable to integrate the visual communication device 162 onto an assembly that is configured for use with the table 24. Accordingly, FIG. 7 depicts a system 180 having a carriage assembly 182 configured for use with the table 24. In some embodiments, the carriage assembly 182 may be a low profile carriage assembly (LPCA) as generally described in U.S. Pat. No. 7,467,004, issued to Calderon et al., on Dec. 16, 2008, entitled “SYSTEM, METHOD AND APPARATUS FOR SURGICAL PATIENT TABLE,” which is incorporated by reference herein in its entirety.

The carriage assembly 182 includes a patient supporting portion 184, which is generally configured to support the patient 26 while interfacing with the table 24. The patient supporting portion 184 may also include features for protecting/housing one or more fiber optic lines, such as fiber optic lines 76 of FIGS. 2-6. In addition to the patient supporting portion 184, the carriage assembly 182 also includes a head portion 186. The head portion 186 is formed into an annular shape that is configured to receive the head of the patient 26 therein. The head portion 186 may allow a technician to place one or more RF receiving coils proximate the head of the patient so as to obtain suitable signal to noise in acquired MR data. In accordance with the present approaches, the head portion 186 of the carriage assembly 182 may also support the visual communication device 162. Of course, the visual communication device 162 may be placed at a proper distance or otherwise be configured so as to provide instructions and/or indications to the patient 26. Further, the close proximity of the visual communication device 162 to the patient's head may allow for certain diagnostics to be performed than would otherwise be appropriate. It should be noted, however, that the visual communication device that is ultimately integrated with the carriage assembly 182 may perform any one or a combination of the functions described above with respect to FIGS. 3-6.

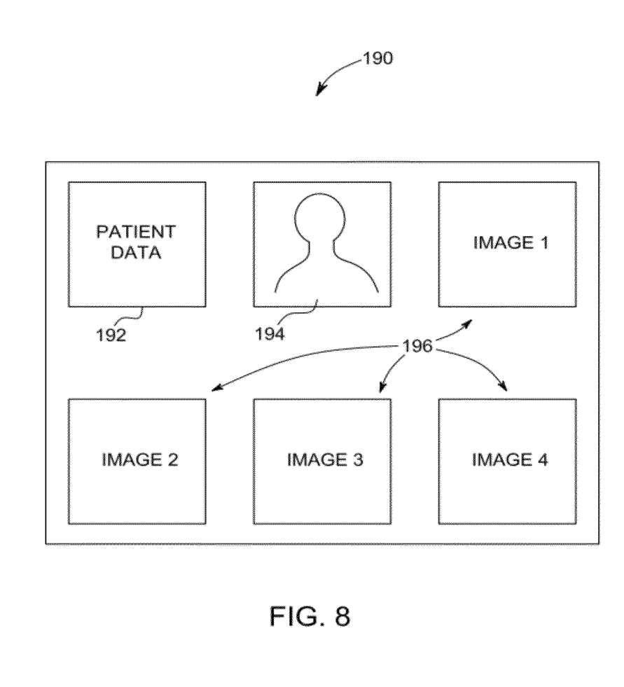

During operation of the MRI system 10 in which the visual communication device in is utilized accordance with the present disclosure, or after such operation, a technician may view a combination of information and images on a screen so as to ascertain image quality, correct patient placement, facilitate future diagnoses, and so forth. An illustration of an embodiment of a collection of such information is provided in FIG. 8. Specifically, FIG. 8 depicts a collection of data 190 including patient data 192 in combination with captured patient images using the visual communication device and the MR scanner. The patient data 192 may include various patient information such as patient biographical data (e.g., height, weight, eye color, hair color), past diagnoses, past imaging sequences performed, physician information, and so on.

Also included in the collection of data 190 is at least one image of the patient 194 that has been captured using the visual communication device in accordance with the present disclosure. The at least one image of the patient 194 may include a real-time visualization of the patient 26, or may simply be a still frame of the patient 26. In combination with the patient data 192 and/or the patient image 194, one or more reconstructed images 196 may be provided. The reconstructed images 196 include at least one MR image that is reproduced from MR data captured using the MR scanner 12, and may also include reconstructed images resulting from data acquired using the same or different imaging modalities, such as computed tomography (CT), positron emission tomography (PET), single photon emission computed tomography (SPECT), radiography, or any combination thereof that may aid in performing diagnostic analysis.

This written description uses examples to disclose the invention, including the best mode, and also to enable any person skilled in the art to practice the invention, including making and using any devices or systems and performing any incorporated methods.

It should also be understood that the various examples disclosed herein may have features that can be combined with those of other examples or embodiments disclosed herein. That is, the present examples are presented in such as way as to simplify explanation but may also be combined one with another. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A magnetic resonance imaging system, comprising:
a scanner having an opening configured to receive a patient and being operable to perform a magnetic resonance imaging sequence; and
a visual communication device disposed in the opening of the scanner and being operable to capture an image of the patient and to transmit the image to circuitry outside of the scanner.

2. The system of claim 1 wherein the circuitry comprises control circuitry connected to the scanner and the visual communication device, and the control circuitry is configured to control the operation of the scanner and the visual communication device.

3. The system of claim 2, wherein the scanner comprises:
a magnet configured to generate a bulk magnetic field to align the spins of gyromagnetic nuclei within the patient;
a series of gradient coils configured to encode the positions of the gyromagnetic nuclei;
a radio frequency coil configured to tip the spins of at least a portion of the encoded gyromagnetic nuclei; and
a receiving coil configured to receive magnetic resonance data resulting from the relaxation of the tipped gyromagnetic nuclei; and
wherein the control circuitry is configured to receive the magnetic resonance data and to generate an image representative of the patient from the data, and the control circuitry is in communication with memory circuitry configured to store the image representative of the patient against the image of the patient captured by the visual communication device.

4. The system of claim 2, wherein the visual communication device is configured to transmit information to the patient while the patient is inside the opening, and the control circuitry is configured to synchronize the magnetic resonance imaging sequence performed by the scanner with the delivery of information to the patient by the visual communication device.

5. The system of claim 4, wherein the visual communication device is optically connected to a light source disposed in an area outside of the scanner via one or more optical fibers.

6. The system of claim 5, wherein the light source comprises a light emitting diode.

7. The system of claim 6, wherein the visual communication device comprises an etched panel configured to receive light from the light source, wherein a first portion of the etched panel illuminates during a first phase of operation of the scanner, and a second portion of the etched panel illuminates during a second phase of operation of the scanner.

8. The system of claim 7, wherein the first phase comprises a patient breath-hold phase and the second phase comprises a patient breathing phase.

9. The system of claim 1, wherein the visual communication device is configured to transmit the image of the patient to a picture and archiving system, a teleradiology system, a hospital information system, or any combination thereof.

10. The system of claim 1, wherein the visual communication device is substantially immune to radiofrequency emissions.

11. The system of claim 1, wherein the visual communication device is optically connected to a receiver disposed in an area outside of the scanner via one or more optical fibers.

12. The system of claim 11, wherein the visual communication device comprises a lens or scope.

13. The system of claim 1, wherein the magnetic resonance imaging system is a closed-bore system or an open system.

14. The system of claim 1, comprising a patient table within the opening of the scanner and being operable to support the patient in the opening, wherein the visual communication device is disposed on the table or at a diametrically opposite end of the opening from the table.

15. A magnetic resonance imaging system, comprising:
a scanner having an opening configured to receive a patient and operable to perform a magnetic resonance imaging sequence; and
a visual communication device disposed in the opening of the scanner and being operable to transmit visual information originating from circuitry outside of the scanner to the opening.

16. The system of claim 15, wherein the circuitry comprises control circuitry connected to the scanner and the visual communication device, and the control circuitry is configured to control the operation of the scanner and the visual communication device.

17. The system of claim 16, wherein control circuitry is configured to synchronize the magnetic resonance imaging sequence performed by the scanner with the delivery of information to the patient by the visual communication device.

18. The system of claim 15, wherein the visual communication device is configured to transmit the information to the patient originating from a teleradiology system and/or a hospital information system.

19. The system of claim 15, wherein the visual communication device is optically connected to a light source disposed in an area outside of the scanner via one or more optical fibers.

20. The system of claim 19, wherein the visual communication device comprises an etched panel configured to receive light from the light source, wherein a first portion of the etched panel illuminates during a first phase of operation of the scanner, and a second portion of the etched panel illuminates during a second phase of operation of the scanner.

21. The system of claim 15, wherein the visual communication device is configured to capture an image of the patient while the patient is in the opening and transmit the image to the circuitry.

22. A magnetic resonance imaging communication system, comprising:
a visual communication device configured to be placed in a patient opening of an magnetic resonance imaging scanner, the visual communication device being operable to transmit visual information originating from circuitry outside of the magnetic resonance imaging scanner to a patient in the patient opening.

23. The system of claim 22, wherein the visual communication device comprises an etched panel configured to receive light from a light source, wherein a first portion of the etched panel illuminates during a first phase of operation of the magnetic resonance imaging scanner and a second portion of the etched panel illuminates during a second phase of operation of the magnetic resonance imaging scanner.

24. The system of claim 22, wherein the circuitry comprises control circuitry configured to control the operation of the magnetic resonance imaging scanner and the visual communication device, and the control circuitry is configured to synchronize operation of the magnetic resonance imaging scanner with the delivery of information to the patient by the visual communication device.

25. The system of claim 22, visual communication device is configured to capture an image of the patient while the patient is in the opening and transmit the image to the circuitry.

26. The system of claim 25, wherein the visual communication device is configured to transmit the image of the patient to a picture and archiving system, a teleradiology system, a hospital information system, or any combination thereof.

27. An image captured using a magnetic resonance imaging communication system, the system comprising:
a visual communication device configured to be placed in a patient opening of an magnetic resonance imaging scanner, the visual communication device being operable to capture the image in the patient opening.
